(19) United States
(12) Patent Application Publication
(10) Pub. No.: US 2022/0008636 A1
(43) Pub. Date: Jan. 13, 2022

(54) APHERESIS TO REMOVE INTERFERING SUBSTANCES

(71) Applicant: Henry J. Smith, Temecula, CA (US)

(72) Inventor: Henry J. Smith, Temecula, CA (US)

(21) Appl. No.: 16/926,771

(22) Filed: Jul. 12, 2020

(51) Int. Cl. A61M 1/36; A61M 1/34

(52) U.S. Cl. CPC A61M 1/362; A61M 1/3486; A61M 1/3679

(57) ABSTRACT

Cancer patients have circulating tumor cell components in their blood. When a therapeutic biologic or biosimilar is administered intravenously into the patient it can bind to these tumor cell components causing adverse side-effects. This invention teaches a targeted apheresis method of removing these interfering tumor cell components by binding them out using an immobilized binding agent contained within an apheresis device, and returning the treated blood back to the patient. Reducing the level of circulating tumor cell components before administering a biologic or biosimilar will increase its safety and efficacy in treating the tumor.

### CROSS-REFERENCE TO RELATED APPLICATIONS

None.

### STATEMENT RE: FEDERALLY SPONSORED RESEARCH/DEVELOPMENT

Not Applicable

### BACKGROUND

There are an increasing number of biological based pharmaceuticals being developed to treat cancer. Many of these are antibodies that target a tumor associated antigen or a cellular receptor or cell-surface marker present on a cancer cell. For example, trastuzumab (Herceptin®) is used to treat breast cancer and stomach cancer; cetuximab (Erbitux®) is used to treat head and neck cancer and colorectal cancer; ramucirumab (Cyramza®) is used to treat stomach cancer, colorectal cancer, liver cancer, and lung cancer; atezolizumab (Tecentriq®) is used to treat stomach cancer, bladder cancer, breast cancer, and lung cancer; nivolumab (Opdivo®) is used to treat bladder cancer, colorectal cancer, kidney cancer, liver cancer, and lung cancer; pembrolizumab (Keytruda®) is used to treat bladder cancer, cervical cancer, kidney cancer, liver cancer, lung cancer, stomach cancer, and head and neck cancer. These anti-tumor antibodies are typically referred to as “biologics”.

It was often observed that when cancer patients received an intravenous injection or infusion of the biologic they frequently experienced fever and chills or nausea immediately after administration of the biologic. For the most part these symptoms were thought to be the result of the way the biologic was being administered too quickly or being too cold; or that the patient was reacting to some of the excipients such as the buffer or stabilizers used. This invention teaches an alternate explanation for this phenomenon and a means to ameliorate the adverse side-effects to the patient.

This invention teaches that many tumors release tumor cells into the blood stream; and/or the tumors can also release nanosized vesicles called exosomes that are composed of part of the tumor cell membrane; and/or tumor cell fragments formed when the tumor cell dies; and/or soluble growth receptors that are enzymatically cleaved off the tumor cells. These circulating tumor cells and/or tumor cell components bear growth receptors identical to the particular growth receptor on the tumor cells that are being targeted by the therapeutic biologic. Therefore when a biologic is administered intravenously into the patient it will immediately bind to the circulating tumor cells or tumor cell components bearing that receptor. The adverse side-effects often observed when a biologic is administered are not due to the mode of administration but are in fact an indication that an immune reaction is occurring.

It would be desirable to develop a method of reducing the level of circulating tumor cell components in order to reduce the adverse side-effects that occur when the therapeutic biologic is administered. A further benefit of removing the interfering tumor cell components is that more of the administered biologic is available to treat the tumor.

This invention teaches a targeted apheresis method of removing the interfering tumor cell components present in the blood of the patient prior to administering the biologic. The art is silent on the use of targeted apheresis to remove circulating interfering tumor cell components in order to improve the safety and efficacy of a therapeutic biologic.

### SUMMARY

Cancer patients have circulating tumor cell components in their blood. When a therapeutic biologic or biosimilar is administered intravenously into the patient it can bind to these tumor cell components causing adverse side-effects. This invention teaches a targeted apheresis method of removing these interfering tumor cell components by binding them out using an immobilized binding agent contained within an apheresis device, and returning the treated blood back to the patient. Reducing the level of circulating tumor cell components before administering a biologic or biosimilar will increase its safety and efficacy in treating the tumor.

### DESCRIPTION OF THE INVENTION

In this invention the term “interfering cell components” is used to describe the presence of tumor cells and/or exosomes, and/or cell fragments, and/or soluble growth receptor circulating in the blood. The term “targeted apheresis” is used to describe the specific removal of only the circulating tumor cells or tumor cell components using an immobilized binding agent contained within in an apheresis device. The term “targeting agent” or “binding agent” is used to describe a molecule such as an antibody or an aptamer or a binding peptide, or a hormone or a cytokine that is capable of specifically binding to its respective target (i.e. antigen or cell receptor). The term “biologic” is typically used to describe a therapeutic antibody that targets an antigen or specific cell receptor present on the tumor cell. It will also include “biosimilars” to the therapeutic antibody. With the development of targeting agents such as aptamers and binding peptides that mimic the targeting capacity of antibodies the term “biologic” is used to include these agents as well. The term “biologic” is also used to include other biological materials such as hormones and cytokines that can be used as binding agents in this invention.

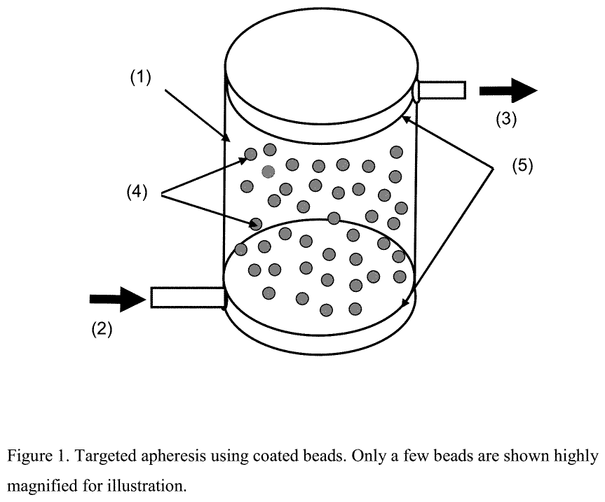

The method of targeted apheresis is based on the well-established methods used in therapeutic apheresis. Typically, the cancer patient's blood is first separated into the plasma fraction and the cellular fraction using differential centrifugation or membrane separation. The plasma is then passed through a targeted apheresis device (FIG. 1). The apheresis device consists of a chamber (1) with an inlet port (2) for plasma to enter, and an outlet port (3) for the plasma to exit. The chamber contains a support matrix coated with a binding agent (e.g. agarose beads (4) coated with an anti-tumor antibody targeting a tumor antigen or a growth factor receptor antibody). There are two porous retaining membranes (5) at the top and bottom of the chamber to retain the beads within the chamber. The pore size of the retaining membranes will retain the beads within the chamber while allowing plasma to pass through. During its passage through the chamber the plasma will come into contact with the immobilized binding agent and the circulating tumor cell components are bound out. The treated plasma is then remixed with the blood cells and returned to the patient.

In one embodiment of this invention whole blood from the patient is passed through a modified targeted apheresis device in which the targeting agent is immobilized on very large beads that are much larger than the red blood cells and white blood cells present in blood. For example the beads are manufactured to be 100-500 um in diameter while the pore size of the retaining mesh or sieve is under 100 um in diameter. This will allow whole blood to flow through the device while retaining the large coated beads within the chamber. When the tumor cell components circulating in the blood come into contact with the immobilized binding agent they will be bound out and the treated blood is then returned to the patient.

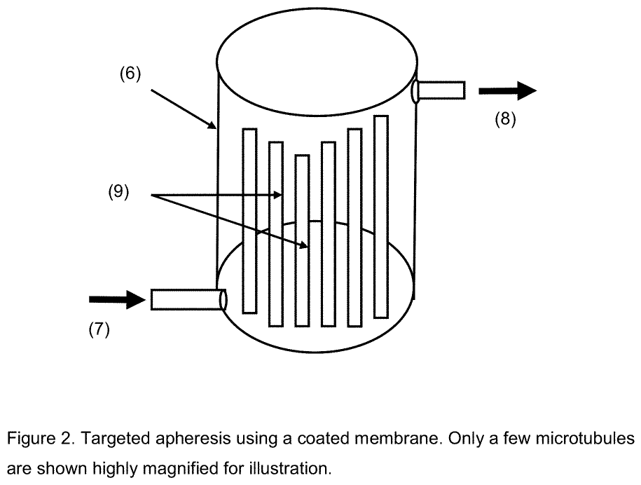

In one embodiment of this invention whole blood or plasma from the patient is passed through a targeted apheresis device (FIG. 2) containing a membrane (9) coated with the binding agent. The membrane may be a permeable, semi-permeable or impermeable membrane whose sole function is to provide a large surface area for attachment of the binding agent. The device consists of a chamber (6) with an inlet port (7) for blood or plasma to enter, and an outlet port (8) for the blood or plasma to exit. To increase the binding capacity of the membrane it may be folded or pleated or be in the form of microtubules (9) of the type seen in dialysis or apheresis cartridges. In contrast to a typical apheresis device there is no pressure differential across the membrane and the blood or plasma is allowed to flow on both sides of the membrane. When blood or plasma comes into contact with the immobilized binding agent on the membrane the tumor cell components are bound out and the treated blood or plasma is returned to the patient.

It will be obvious to one of skill in the art that other device configurations and other support matrixes can be employed without departing from the spirit and scope of this invention. Said modifications are therefore considered to lie within the scope of this invention.

In one embodiment of this invention the targeting agent is an anti-tumor antibody. For example, the anti-tumor antibody targets the over-expressed human epidermal growth factor receptor 2 (HER-2) on cancer cells of certain breast cancers. In this invention the term “antibody” will include polyclonal, monoclonal and recombinant antibodies, and the binding site fragments of those antibodies. Polyclonal antibodies are produced by immunizing animals with a tumor associated antigen and collecting the serum. The antiserum is processed using established methods such as salt-fractionation, gel chromatography and affinity purification to prepare a purified anti-tumor antibody. Monoclonal antibodies are prepared using hybridoma technology using mice, rabbit, human or other cell lines. When prepared in other species they are often “humanized” by replacing certain components of the monoclonal antibody with human components. Recombinant antibodies are produced using genetic engineering techniques in which the genetic code for the antibody is identified and then expressed in genetically modified bacteria, or fungi, or insect and mammalian cells lines. These and other methods of producing purified anti-tumor antibodies are well-known to those of skill in the art and are considered to lie within the scope of this invention. In this invention the term antibody refers to the whole antibody molecule, and/or the binding fragments Fab and F(ab)2; and/or to recombinant binding proteins such as scFv.

It will be also obvious to one of skill in the art that there other types of targeting agents such as aptamers and binding peptides that mimic the binding capacity of antibodies. Therefore aptamers and binding peptides are considered to be functional equivalents of an antibody; and can be used in lieu of an antibody in the targeted apheresis device without departing from the spirit and scope of this invention.

In one embodiment of this invention the targeting agent is an anti-tumor aptamer. For example, an aptamer that targets human epidermal growth factor receptor 2 (HER-2). Aptamers are small (i.e. 40-100 bases), synthetic single-stranded oligonucleotides (ssDNA or ssRNA) that can specifically recognize and bind to virtually any kind of target, including ions, whole cells, drugs, toxins, low-molecular-weight ligands, peptides, and proteins. Each aptamer has a unique configuration as a result of the composition of the nucleotide bases in the chain causing the molecule to fold in a particular manner. Because of their folded structure each aptamer will bind selectively to a particular ligand in a manner analogous to an antibody binding to its antigen. Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand. In order to improve stability against nucleases found in vivo the oligonucleotides may be modified to avoid nuclease attack. They may for example be synthesized as L-nucleotides instead of the natural D-nucleotides and thus avoid degradation from the natural nucleases. In this invention the term “aptamer” is used to describe all varieties of aptamers including single strand RNA aptamers (ssRNA) and single strand DNA aptamers (ssDNA). It also includes all modifications such as adding or substituting different bases into the chain and/or altering its structure (e.g. branched aptamer).

In one embodiment of this invention the targeting agent is an anti-tumor binding peptide. For example, a binding peptide that targets human epidermal growth factor receptor 2 (HER-2). Binding peptides consist of a chain of aminoacids that fold in such a manner that their configuration makes them capable of binding to antigens in a manner that mimics the binding of an antibody to its antigen. There are various well-known methods for preparing synthetic or biological peptide libraries composed of up to a billion different sequences, and for identifying a particular peptide sequence that will target a particular antigen. In this invention the term binding peptide is used to describe all varieties of binding peptides capable of binding to the target antigen irrespective of the molecular composition of the peptide chain.

In one embodiment of this invention the targeting agent does not target the same antigen as the therapeutic biologic, but instead targets other antigens that are present on the same tumor cell component. For example, circulating breast cancer cell fragments may have in addition to growth factor receptors other receptors such as hormone receptors or cell-surface markers. Therefore it is possible to employ a binding agent that targets a hormone receptor or cell-surface marker and by binding these out this will also cause the tumor cell component bearing the growth factor receptor targeted by the therapeutic biologic to be also bound out.

In one embodiment of this invention an antibody that can target either the same antigen as the therapeutic antibody or a different antigen on the same tumor cell component is used as the binding agent.

In one embodiment of this invention an aptamer that can target either the same antigen as the therapeutic antibody or a different antigen on the same tumor cell component is used as the binding agent.

In one embodiment of this invention a binding peptide that can target either the same antigen as the therapeutic antibody or a different antigen on the same tumor cell component is used as the binding agent.

It will also be obvious to those of skill in the art that targeting ligands such as hormones, growth factors, cytokines and the like can similarly be used to prepare tumor targeting agents.

In one embodiment of this invention a hormone such as estrogen is used as the immobilized binding agent to remove circulating tumor cell components that bear estrogen receptors.

In one embodiment of this invention a hormone such as progesterone is used as the immobilized binding agent to remove circulating tumor cell components that bear progesterone receptors.

Typically the binding agent is immobilized on cross-linked agarose beads. However, there are many other types of support matrixes that may be used instead. These include beads of other chemical composition such as silica beads or acrylic beads or cellulose beads and the like. Or the support could be a membrane to which the binding agent is attached. These and other types of supports are well-known to those of skill in the art.

The methods of attaching the binding agent to the support matrix are known to those of skill in the art. One method is by passive adsorption of the binding agent to the matrix. In most cases the binding agent is preferably covalently attached to the matrix.

The following examples are provided as a means of illustration, and not limitation, on how the apheresis procedure works. It will be obvious to those of skill in the art that the same principles taught in this invention can apply to a wide variety of tumors. Any apheresis process based on these principles is therefore considered to lie within the spirit and scope of this invention.

### Example 1

Removal of interfering tumor cell components using a binding agent that targets the same target as the biologic. Some breast cancers are composed of cancer cells that have over-expressed growth factor receptors called Epidermal Growth Factor Receptor 2 (HER2) on the cell surface. Breast cancer patients who have this type of breast cancer are treated with a biologic (e.g. trastuzumab) that targets these receptors and blocks them from receiving growth signals; and this inhibits tumor growth. This invention teaches that there are tumor cell components circulating in the blood that bear epidermal growth factor receptor 2. Therefore when a biologic that targets epidermal growth factor receptor 2 is administered to the patient it would bind to the circulating cell components and cause an adverse side-effect. It would also mean that there is less biologic available to treat the tumor.

The interfering tumor cell components can be removed using targeted apheresis employing an immobilized binding agent that targets the same antigen as the biologic. For example the biologic trastuzumab is attached to agarose beads and the coated beads are placed in an apheresis device. When blood or plasma comes into contact with the immobilized binding agent the interfering tumor cell components are bound out and the treated blood is returned to the patient. There is therefore a reduced level of interfering tumor cell components present in the patient's blood to elicit an adverse side-effect when the therapeutic biologic is administered.

Other examples of using the same biologic or biosimilar to be the binding agent as the therapeutic biologic are: cetuximab (Erbitux®); ramucirumab (Cyramza®); atezolizumab (Tecentriq®); nivolumab (Opdivo®) and pembrolizumab (Keytruda®).

In one embodiment of this invention the binding agent is an aptamer that targets the same target antigen as the biologic.

In one embodiment of this invention the binding agent is a binding peptide that targets the same target as the biologic.

### Example 2

Removal of interfering tumor cell components using a binding agent that targets a different target from the biologic. For example, breast cancer cells have Epidermal Growth Factor receptors and the therapeutic biologic being used targets epidermal growth factor receptors (e.g. cetuximab). It is possible to remove interfering tumor cell components that bear epidermal growth factor receptors using a binding agent that targets an antigen that is not epidermal growth factor receptor. For example the binding agent could target estrogen receptor and achieve the same result. The reason is that the tumor cell can have a variety of different receptors and surface marker proteins on the cell membrane. These could include growth factor receptors and hormone receptors. Therefore the tumor cell components could carry a variety of receptors that could be targeted and bound out. For example, if the immobilized binding agent bound out estrogen receptors and if the epidermal growth factor receptor was also present on the same tumor cell component then it would also be bound out.

### Example 3

Removal of interfering tumor cell components using a ligand as the immobilized binding agent.

For example, the immobilized binding agent could be Epidermal Growth Factor. This will bind out tumor cell components bearing Epidermal Growth Factor Receptors. Or the immobilized binding agent could be a hormone such as estrogen. This will bind out tumor cell components bearing estrogen receptors; and if the tumor cell component also bears Epidermal Growth Factor Receptors these will be bound out as well.

The same principle will apply to using immobilized progesterone as the binding agent in estrogen negative progesterone positive breast cancer cases. This will bind out tumor cell components bearing progesterone receptors and if the tumor cell component also bears Epidermal Growth Factor Receptors these will be bound out as well.

It will be obvious to one of skill in the art that other hormones or cell receptors or cell surface markers that are present on the same cell component as the antigen that the biologic is targeting can be similarly employed to bind out the tumor cell component.

This invention teaches the removal of interfering tumor cell components in the blood using targeted apheresis. Reducing the amount of interfering tumor cell components in the blood will result in a reduction in the adverse side-effects that occur when a therapeutic biologic is administered. The removal of the interfering tumor cell components will also result in more of the biologic or biosimilar being available to treat the tumor. It will be obvious to one of skill in the art that there are various modifications that can be made to the general principles disclosed in this invention and how they can be applied to treat a variety of tumors. Said modifications that are made as a result of the teachings in this invention are therefore considered to lie within the spirit and scope of this invention.

1. A targeted apheresis method of removing circulating tumor cell components present in blood; wherein the removal of said tumor cell components would reduce the adverse side-effects commonly encountered when a therapeutic biologic or biosimilar is administered to the patient; and wherein said treatment will increase the bioavailability of the biologic or biosimilar to treat the tumor.

2. A targeted apheresis method according to claim 1 wherein the circulating tumor cell components are bound out using an immobilized binding agent that targets the same antigen or growth factor receptor as the biologic or biosimilar.

3. A targeted apheresis method according to claim 1 wherein the circulating tumor cell components are bound out using an immobilized binding agent that targets a different antigen or receptor than that targeted by the biologic or biosimilar.

4. A targeted apheresis method according to claim 1 wherein the tumor cell components are bound out using an immobilized binding agent that is either an anti-Epidermal Growth Factor Receptor antibody, or an anti-Epidermal Growth Factor Receptor aptamer; or an anti-Epidermal Growth Factor Receptor binding peptide.

5. A targeted apheresis method according to claim 1 wherein the tumor cell components are bound out using an immobilized binding agent that is either an anti-Epidermal Growth Factor Receptor 2 (HER-2) antibody, or an anti-Epidermal Growth Factor Receptor 2 (HER-2) aptamer; or an anti-Epidermal Growth Factor Receptor 2 (HER-2) binding peptide.

6. A targeted apheresis method according to claim 1 wherein the tumor cell components are bound out using an immobilized binding agent that is either estrogen or progesterone.

7. A targeted apheresis method according to claim 1 wherein the tumor cell components are bound out using a binding agent that is immobilized on a support matrix that is either in the form of beads, or as a membrane with a large surface area.
